(19) United States
(12) Patent Application Publication
(10) Pub. No.: US 2005/0003499 A1
(43) Pub. Date: Jan. 6, 2005

(54) Ion-exchange filtration of fermentation broth

(76) Inventors: Vilmos Keri, Debrecen (HU); Istvan Melczer, Debrecen (HU); Lajos Deak, Debrecen (HU); Krisztian Szeles, Hajduszoboszlo (HU)

Correspondence Address: KENYON & KENYON, One Broadway, New York, NY, 10004-1050, US

(21) Appl. No.: 10/865,590

(22) Filed: Jun. 9, 2004

Related U.S. Application Data
(60) Provisional application No. 60/477,102, filed on Jun. 9, 2003.

(51) Int. Cl. C12P 7/62

(52) U.S. Cl. 435/135

(57) ABSTRACT

The invention encompasses a process for purifying a fermentation broth by providing a fermentation broth; adjusting the pH of the fermentation broth; isolating a filtrate from the fermentation broth; and passing the filtrate through a cation-exchange resin to obtain a purified filtrate.

### FIELD OF THE INVENTION

The present invention is directed to methods for isolating and purifying an active agent within a fermentation broth of a HMG-CoA reductase inhibitor.

### BACKGROUND OF THE INVENTION

Complications of cardiovascular disease, such as myocardial infarction, stroke, and peripheral vascular disease account for half of the deaths in the United States. A high level of low density lipoprotein (LDL) in the bloodstream has been linked to the formation of coronary lesions which obstruct the flow of blood and can rupture and promote thrombosis. Goodman and Gilman, THE PHARMACOLOGICAL BASIS OF THERAPEUTICS, p. 879 (9th ed., 1996). Reducing plasma LDL levels has been shown to reduce the risk of clinical events in patients with cardiovascular disease and in patients who are free of cardiovascular disease but who have hypercholesterolemia. Scandinavian Simvastatin Survival Study Group, 1994; Lipid Research Clinics Program, 1984a, 1984b.

Statin drugs are currently the most therapeutically effective drugs available for reducing the level of LDL in the blood stream of a patient at risk for cardiovascular disease. This class of drugs includes, inter alia, compactin, lovastatin, simvastatin, pravastatin and fluvastatin. The mechanism of action of statin drugs has been elucidated in some detail. The statin drugs disrupt the synthesis of cholesterol and other sterols in the liver by competitively inhibiting the 3-hydroxy-3-methyl-glutaryl-coenzyme A reductase enzyme (“HMG-CoA reductase”). HMG-CoA reductase catalyzes the conversion of HMG-CoA to mevalonate, which is the rate determining step in the biosynthesis of cholesterol. Consequently, HMG-CoA reductase inhibition leads to a reduction in the rate of formation of cholesterol in the liver.

Pravastatin is the common medicinal name of the chemical compound [1S-[1α(β*, δ*)2α,6α,8β(R*),8aα]]-1,2,6,7,8,8a-hexahydro-β,δ,6-trihydroxy-2-methyl-8-(2-methyl-1-oxobutoxy)-1-naphthalene-heptanoic acid. The molecular structure of pravastatin in free acid form is represented by Formula (I):

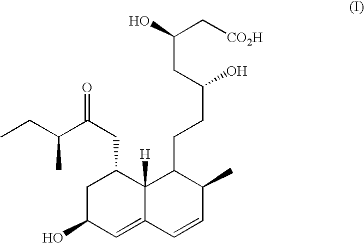

Pravastatin possesses an alkyl chain that is terminated by a carboxylic acid group and that bears two hydroxyl groups at the β and δ positions with respect to the carboxylic acid group, which may close into a lactone. The alkyl chain is the portion of the molecule that binds to HMG-CoA reductase. The carboxylic acid group and the hydroxyl group at the δ position are prone to lactonization. Compounds that form a lactone, like the statins, may exist either in the free acid form or the lactone form or in an equilibrium mixture of both forms. Compounds that form lactones cause processing difficulties during the manufacture of statin drugs because the free acid and the lactone forms of the compounds have different polarities. One method of purifying one form will remove impurities but also is likely to remove the other form thereby resulting in a lower overall yield. Consequently, great care must be exercised when handling lactonizable compounds to isolate them in high yield.
Lovastatin and its analogs, e.g. simvastatin, are potent antihypercholesterolemic agents that function by limiting cholesterol biosynthesis. Lovastatin is one of the most important known cholesterol lowering agents. Lovastatin is also known as mevinolin or monacolin K and is chemically known as: β,δ-dihydroxy-7-[1,2,6,7,8,8a-hexahydro-2,6-dimethyl-8-(2-methyl-butyryloxy)-1-napthalen-1-yl]-heptanoic acid δ-lactone, i.e., the lactone form of lovastatin is shown below:

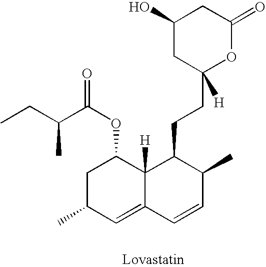

Lovastatin and its analogs inhibit the enzyme 3-hydroxy-3-methyl-glutarylcoenzyme A reductase (“HMG-CoA reductase”). HMG-CoA reductase catalyzes the formation of mevalonic acid, an early intermediate of cholesterol biosynthesis. Lovastatin is specifically advantageous because, as a result of its application, biosynthetic intermediates that have a toxic steroid skeleton, formed at a later stage of biosynthesis, fail to accumulate. Lovastatin also increases the number of LDL-receptors at the surface of the cell membrane, which remove the LDL cholesterol circulating in the blood, thereby inducing the lowering of blood plasma cholesterol level.

Lovastatin is routinely produced via fermentation. GB 2,046,737 discloses that lovastatin can be produced by some strains belonging to the Monascus genus, e.g., by M ruber 1005 cultivated between 7° C. and 40° C. As a culture medium, an aqueous solution of glucose, peptone, corn steep liquor and ammonium chloride was used. The fermentation was carried out for 10 days in aerobic conditions, and 87 mg lovastatin was obtained from the filtrate of 5 liters of broth.

The known methods for isolating a statin from a fermentation broth, however, are ill-suited for isolating pharmaceutically acceptable levels of purity, or alternatively, the methods require economically impractical chromatographic separation to achieve high purity. The present invention meets the need in the art for an efficient and practical method for isolating HMG-CoA reductase inhibitors from a fermentation broth in high purity, in high yield, and/or on a preparative scale.

### SUMMARY OF THE INVENTION

The invention encompasses processes for purifying a fermentation broth comprising providing a fermentation broth; adjusting the pH of the fermentation broth to an alkaline pH; isolating a filtrate from the fermentation broth; and passing the filtrate through a cation-exchange resin to obtain a purified filtrate. In one embodiment, the process further comprises reducing the volume of the purified filtrate by nanofiltration, wherein the step comprises passing the purified filtrate through a microfiltration membrane.

The fermentation broth may be a fermentation broth of a HMG-CoA reductase inhibitor, wherein the HMG-CoA reductase inhibitor may be compactin, lovastatin, or pravastatin. Preferably, the pH of the fermentation broth is adjusted to a pH of about 8 to about 10.

In yet another embodiment, the isolating step is performed with a centrifuge or a filter to remove mycelium from the fermentation broth. Preferably, the isolation is conducted using a microfiltration membrane. Typically, the isolation may be carried out at a temperature of about 10° C. to about 90° C., and preferably, the temperature is about 20° C. to about 65° C.

Passing the filtrate through the cation exchange resin removes cations that may induce the active ingredient to precipitate. In one embodiment, the cation exchange resin is a weak acid resin, a strong acid resin, a chelating cation-exchanger, or a combination thereof. The cation exchange resin has a mono-valent cation including, but not limited to, H⁺, Li⁺, Na⁺, K⁺, or NH₄⁺ ions, and the cation exchange resin removes magnesium and/or calcium ions. In yet another embodiment, the filtrate pH is adjusted to a pH of about 7.0 to about 14.0 prior to passing the filtrate through the cation exchange resin.

### DETAILED DESCRIPTION OF THE INVENTION

The invention encompasses a process for isolating HMG-CoA reductase inhibitor from a fermentation broth comprising the use of a cation-exchange resin to remove cations that may induce precipitation of the active ingredient during concentration of the purified filtrate. Also, the use of the cation-exchange resin increases the rate of nanofiltration, and/or prevents membrane fouling during nanofiltration. Overall, the filtered concentrated broth or purified filtrate facilitates the isolation of the active ingredient by decreasing the volume of material manipulated during the isolation steps necessary to obtain the active ingredient and removing ions that may induce precipitation of the active ingredient, which may complicate the isolation process.

The process of the invention comprises adjusting the pH of the fermentation broth to an alkaline pH; isolating a filtrate from the fermentation broth; and passing the filtrate through a cation-exchange resin to obtain a purified filtrate. The process may further comprise a step for reducing the volume of the purified filtrate by nanofiltration.

The fermentation broth may be any broth of a HMG-CoA reductase inhibitor. Optionally, the HMG-CoA reductase inhibitor is compactin, lovastatin, or pravastatin. Typically, the pH of the broth is adjusted to an alkaline pH of about 7 to about 14. Preferably, the broth pH is adjusted to about 8 to about 10. More preferably, the broth pH is adjusted to about 8 to about 8.5 or to about 9.2 to about 9.6. One of ordinary skill in the art can easily determine how to adjust the pH to obtain the desired range. For example, a solution of NaOH may be added to the solution to adjust the pH. Other examples of suitable bases include, but are not limited to, KOH, NH₄OH, and other solutions that make the pH alkaline.

The isolation step comprises removing the mycelium from within the broth. Generally, the isolation of the filtrate from the fermentation broth can be carried out using a centrifuge or alternatively, a filter. Suitable centrifuges include, but are not limited to, a solid bowl centrifuge. Suitable filters include, but are not limited to, vacuum drum filters, ceramic membrane filters, nuts filters, or any other filter that can remove the mycelium from the fermentation broth. The pore sizes of the membranes may be of any size, e.g. 5 μm to 0.05 μm. Preferably, the isolation is conducted using a microfiltration membrane. When the isolation is conducted using a microfiltration membrane, the membrane may have a 0.05 μm, 0.1 μm, or 0.2 μm pore size. A microfiltration membrane may be a ceramic membrane, a membrane produced by polymerization, etc. The isolation step may be repeated as necessary to remove a suitable amount of mycelium.

The isolation may be carried out at any temperature as long as the active substance remains stable. Typically, the isolation may be carried out at a temperature of about 10° C. to about 90° C. Preferably, the isolation temperature is about 20° C. to about 65° C.

Passing the filtrate from the isolation step through a cation exchange resin may be conducted using techniques commonly known in the art. Passing the filtrate through the cation exchange resin removes cations that may induce the active ingredient to precipitate, increases the rate of nanofiltration, and/or prevents membrane fouling during nanofiltration. Passing the filtrate through the cation exchange resin removes cations, such as magnesium or calcium, that may induce the active ingredient to precipitate. The cation resin may be at least one of a weak acid resin, a strong acid resin, or a chelating resin. Also, there may be more than one cation resin column wherein the columns are placed in consecutive sequence. The cation exchange resin may be any weakly acidic resin including, but not limited to, the cation resins in the form of hydrogen, ammonia, lithium, sodium, potassium, or any mono-valent cation, preferably, in ammonia form. Exemplary commercially available cation resins include those sold by Sybron Chemicals Inc. (Pittsburgh, Pa. 15205) such as Lewatit CNP 80; those sold by Rohm & Haas Co. (Philadelphia, Pa. 19106) such as IMAC® HP 333, IMAC® HP 336, Amberlite® CG 50, or Amberlite® IRC 86; those sold by The Purolite Company (Bala Cynwyd, Pa. 19106) such as Purolite® C104, Purolite® C106, Purolite® C107, or Purolite® C115; and those sold by Mitsubishi Kasei Corporation, Japan, such as DAION™ WK types, and the like. Strong acid cation resins are also suitable, including, but not limited to, Amberjet® 1200, C20N/2014, IMAC® C16P, Amberlite® IRN, Amberlite® IRP, Purolite® C/5GC, Purolite® NRV, DAION™ SK, and the like. Chelating resins may also be used such as, Duolite® C467, Amberlite® IRC748, Purolite® S, Duolite® CD types, and the like.

The cation exchange resin may be washed with water and the washings added to the filtrate.

The step for reducing the volume of the purified filtrate comprises passing the purified filtrate through nanofiltration membrane. The nanofiltration membrane may have a cut-off of 200 Daltons or 300 Daltons (D). When necessary, pressure may be applied during the filtrate input in an amount sufficient to encourage the flow of filtrate. Typically, the pressure is about 25 bar. Nanofiltration membranes which can be used include those sold by Koch Membranes Systems (Wilmington, Mass. 01887) such as MPS-34 and MPT-34.

Prior to passing the filtrate through the cation resin, the pH of the filtrate should be in a pH range of about 7.0 to about 14.0, preferably from about 8.0 to about 10. If during the process the pH must be stabilized within a particular range then any base or acid may be used. Preferably, when using a base NaOH (20%), KOH, NH₄OH, and other solutions that make the pH alkaline are used.

Having described the invention with reference to certain preferred embodiments, other embodiments will become apparent to one skilled in the art from consideration of the specification. The invention is further defined by reference to the following examples describing in detail the methods of use of the invention. It will be apparent to those skilled in the art that many modifications, both to materials and methods, may be practiced without departing from the scope of the invention.

### EXAMPLES

### Example 1

### Pravastatin

The pH of a pravastatin fermentation broth (100 kg, 705 g active substance) was adjusted to a pH of 8.0 to 8.5 using a sodium hydroxide solution (20% by mass). The pH adjusted broth was heated to a temperature of 60° C. to 65° C. and filtered with a ceramic membrane (50 nm) to obtain a concentrated broth (60 l). The concentrated broth was filtered further while simultaneously diluting the solution with water, wherein the water did not contain calcium or magnesium ions. Thereafter, the filtrate was collected (400 l). The filtration rate commenced at 138 l/m² h to a final rate of 202 l/m² h. Pravastatin was collected in 91% yield.

The filtrate was purified using a cation-exchange resin column made by placing 6 l of cation-exchange resin in ammonia form, Lewatit CNP 80, into a column 1 m×10 cm. The filtrate was passed through a resin bed at a flow rate of 23 l/h at about 25° C. The resin was washed with 20 l of water whereupon the filtrate and washings were combined (417 l). The yield from the ion exchange was calculated to be 100%.

The purified filtrate was concentrated using a nanofiltration membrane of 200 D cut-off and a tubular membrane MPT-34. The concentration was conducted at a temperature of 65° C., pH of 8.0 to 8.5, and until the volume was reduced to 40 l. The average filtration rate was 34 l/m² h and approximately 2% of the active substance was found in the permeate. The process was repeated using spiral membrane MPS-34, whereupon 3% of the active substance was found in the permeate.

### Example 2

### Compactin Purification

Compactin fermentation broth (150 kg) was diluted with water (30 l, total calcium and magnesium content was 2.4 mmol/l) and the pH adjusted to 9.2 to 9.6 using sodium hydroxide (20%). The broth was filtered using a nuts filter at a temperature of 20° C. to 30° C.; the filtered mycelium was washed with water, suspended at alkaline pH, and filtered again. The collected filtrate was 330 l containing 973 g of active substance.

The filtrate was purified using a cation-exchange resin column made by placing 7 l of cation-exchange resin in ammonia form, Lewatit CNP 80, into a column 1 m×10 cm. The filtrate was passed through the resin bed at a flow rate of 13 l/h at about 30° C. to 33° C. The resin was washed with 10 l of water whereupon the purified filtrate and washings were combined (340 l). The yield from the ion exchange was calculated to be 99%.

The purified filtrate was concentrated using a nanofiltration membrane of 300 D cut-off and a spiral membrane MPS-34. The concentration was conducted at a temperature of 65° C., pH of 9.2 to 9.6, and until the volume was reduced to 70 l. When necessary a pressure of 25 bar was applied at the input of the membrane. The average filtration rate was 14 l/m² h and approximately 3% of the active substance was found in the permeate. The process was repeated using tubular membrane MPT-34, whereupon 2% of the active substance was found in the permeate.

### Example 3

### Lovastatin Purification

The pH of a lovastatin fermentation broth (100 m³) was adjusted to a pH of 9.2 to 9.6 using a sodium hydroxide solution (20% by mass). The pH adjusted broth was stirred for 2 h, and filtered using a vacuum drum filter at a temperature of 30° C. to 35° C. The filtered mycelium was washed with alkaline water, suspended in alkaline pH, and filtered again to obtain a concentrated broth (220 m³).

The filtrate was purified using a cation-exchange resin column made by placing 14 l of cation-exchange resin in ammonia form, Lewatit CNP 80, into two columns 1 m×10 cm connected in series. The filtrate (840 l) was passed through the resin bed at a flow rate of 23 l/h at about 30° C. to 33° C. The resin was washed with 20 l of water whereupon the purified filtrate and washings were combined. The yield from the ion exchange was calculated to be 99%.

The purified filtrate was concentrated using a nanofiltration membrane of 200 D cut-off and a tubular membrane MPT-34. The average filtration rate was 28 l/m² h and approximately 2% of the active substance was found in the permeate. The concentration was conducted at a temperature of 65° C., pH of 9.2 to 9.6, and until the volume was reduced to 70 l. When necessary a pressure of 25 bar was applied at the input of the membrane. The process was repeated using spiral membrane MPS-34, whereupon 3% of the active substance was found in the permeate.

### Example 4

### Solid Bowl Centrifuge

One part lovastatin fermentation broth was diluted with half part water. The pH of the diluted fermented broth was adjusted to 8.0 using sodium hydroxide solution. The pH-adjusted fermented broth was passed through a solid bowl centrifuge, an OV-34 produced by the Hungarian company BVG. The applied flow rate was 360 liters/hour. The solid bowl centrifuge separated the pH adjusted fermentation broth into clear filtrate and into wet mycelium. The wet mycelium contained 76% water by weight. The produced filtrate was suitable for ion-exchange chromatography.

1. A process for purifying a fermentation broth comprising:
providing a fermentation broth;
adjusting the pH of the fermentation broth to alkaline pH;
isolating a filtrate from the fermentation broth; and
passing the filtrate through a cation-exchange resin to obtain a purified filtrate.

2. The process according to claim 1, further comprising repeating the isolation step.

3. The process according to claim 1, wherein the isolation step is carried out at a temperature of about 20° C. to about 65° C.

4. The process according to claim 1, further comprising reducing the volume of the purified filtrate by nanofiltration.

5. The process according to claim 1, wherein the fermentation broth is a fermentation broth of a HMG-CoA reductase inhibitor.

6. The process according to claim 5, wherein the HMG-CoA reductase inhibitor is compactin, lovastatin, or pravastatin.

7. The process according to claim 1, wherein the pH of the fermentation broth is adjusted to a pH of about 7 to about 14.

8. The process according to claim 1, wherein the pH of the fermentation broth is adjusted to a pH of about 8 to about 10.

9. The process according to claim 8, wherein the pH of the fermentation broth is adjusted to a pH of about 8.0 to about 8.5.

10. The process according to claim 8, wherein the pH of the fermentation broth is adjusted to a pH of 9.2 to about 9.6.

11. The process according to claim 1, wherein the isolating step is performed with a centrifuge or a filter.

12. The process according to claim 11, wherein the filter is done with a microfiltration membrane.

13. The process according to claim 12, wherein the microfiltration membrane may have a pore size of 0.05 μm, 0.1 μm, or 0.2 μm.

14. The process according to claim 11, wherein the centrifuge is a solid bowl centrifuge to remove mycelium from the fermentation broth.

15. The process according to claim 11, wherein the filter is a vacuum drum filter, a ceramic membrane filter, or a nuts filter to remove mycelium from the fermentation broth.

16. The process according to claim 1, wherein passing the filtrate through the cation exchange resin removes cations that may induce the active ingredient to precipitate.

17. The process according to claim 1, wherein the cation exchange resin is a weak acid resin, a strong acid resin, a chelating cation-exchanger, or a combination thereof.

18. The process according to claim 1, wherein the resin has mono valent cations.

19. The process according to claim 18, wherein the cation exchange resin has H⁺, Li⁺, Na⁺, K⁺, or NH₄⁺ ions.

20. The process according to claim 1, wherein the cation exchange resin removes magnesium and/or calcium ions.

21. The process according to claim 1, wherein the filtrate has a pH of about 7.0 to about 14.0 prior to passing the filtrate through the cation exchange resin.
